(19) United States
(12) Patent Application Publication
(10) Pub. No.: US 2015/0238137 A1
(43) Pub. Date: Aug. 27, 2015

(54) METHOD AND SYSTEM FOR DETECTING SLEEP DISTURBANCES

(71) Applicant: HypnoCore Ltd., Petach-Tikva (IL)

(72) Inventors: Shulamit Eyal, Givat Shmuel (IL); Armanda Lia Baharav, Tel-Aviv (IL); Shaul Gal-Oz, Palo Alto, CA (US)

(21) Appl. No.: 14/630,696

(22) Filed: Feb. 25, 2015

Related U.S. Application Data
(60) Provisional application No. 61/944,098, filed on Feb. 25, 2014.

(51) Int. Cl. A61B 5/00; A61B 5/024; A61B 5/11

(52) U.S. Cl. CPC A61B 5/4815; A61B 5/4812; A61B 5/4809; A61B 5/7246; A61B 5/6898; A61B 5/11; A61B 5/024; A61B 5/7282; A61B 2560/0242; A61B 2562/162

(57) ABSTRACT

A method of detecting sleep disturbances is disclosed. The method comprises: sensing from a body part of a subject a subject signal indicative of a sleep state of the subject; sensing from an environment containing the subject an environment signal indicative of a state of the environment; identifying awakening and/or arousal events based on the subject signal, and changes in the environment state based on the environment signal; and correlating the awakening and/or arousal events to the changes in the environment state, thereby detecting the sleep disturbances.

### RELATED APPLICATION

This application claims the benefit of priority under 35 USC 119(e) of U.S. Provisional Patent Application No. 61/944,098 filed on Feb. 25, 2014, the contents of which are incorporated herein by reference in their entirety.

### FIELD AND BACKGROUND OF THE INVENTION

The present invention, in some embodiments thereof, relates to sleep analysis and, more particularly, but not exclusively, to a method and system for detecting sleep disturbances.

Sleep represents an essential part of human life and sleep disturbance or insufficient sleep has important negative effects on physical and cognitive performance as well as on general health. Sleep disorders and insufficient sleep are connected with health issues, memory and reaction time, and have also negative impact on work and traffic accidents. Even life span is influenced by sleep, with people with insufficient sleep having a shorter life expectancy.

Typically, an individual has four to six sleep cycles per night, each between 60 and 120 minutes in length and comprising of different proportions of rapid eye movement (REM) sleep and non-REM sleep. Each sleep cycle typically begins with non-REM sleep and ends with REM sleep. The first half of the night contains most of slow wave sleep (SWS), whereas REM sleep is most prominent in the second half. SWS is considered the deepest and most restorative of sleep stages during which there is a reduction in heart rate, blood pressure, sympathetic nervous activity, and brain glucose metabolism, and increase in vagal tone. Hypothalamo-pituitary-adrenal activity is suppressed during SWS and increased during REM sleep.

Poor sleep quality resulting from sleep fragmentation alters the proportion of REM, non-REM and SWS obtained per night. Frequent arousals can disrupt the sequence of sleep stages with patterns of awakening that limit the amount of REM and SWS. Chronic inadequate sleep also manifests in patients who suffer from an inability to easily fall asleep or maintain sleep throughout the night.

Numerous techniques for sleep analysis have been developed.

U.S. Pat. No. 7,623,912 to Akselrod et al., the contents of which are hereby incorporated by reference, describes a technique for determining sleep stages from an ECG signal. A series of cardiac R-R intervals is extracted from the ECG signal and decomposed by a time-frequency decomposition. The time-frequency decomposition is used for determine SWS period and sleep-onset period. EMG parameters are also extracted from the ECG signal and are used for determining REM period. Akselrod et al., also discloses a Poincare plot of the R-R intervals and describes technique for determining REM sleep based on the plot.

International Publication No. WO2011/114333 to Eyal et al., the contents of which are hereby incorporated by reference, discloses a method of analysis which comprises receiving a signal indicative of heart beats of a sleeping subject, extracting from the signal a series of inter-beat intervals, calculating a Poincare parameter characterizing a Poincare plot of the IBI series, and using the Poincare parameter to determine a REM sleep of the sleeping subject.

U.S. Published Application No. 20110137188 to Kuo et al., discloses a sleep assistant system, which comprises an electrocardiogram collector, a heartbeat recognition device, a frequency domain analysis device, and a nerve active judgment device. The nerve active judgment device determines whether cardiac cycle sequences obtained by the heartbeat recognition device, and power of different frequency obtained by the frequency domain analysis device have any effect on disrupted sleep.

U.S. Published Application No. 20130344465 to Dickinson et al., discloses an automated sleep coaching system that provides a personalized sleep coaching plan for a particular user. The system comprises a headband-mounted first sensor that senses a physiological signal associated with a sleeping user. Factors such as light, sound, temperature, and humidity are tracked over time and compared to a user's sleep over the course of a night or compared over many nights. The sensor transmits the sensed physiological signal to a base station that processes the signal and transmits the resulting data to a host computer. The host computer also receives indications of user sleeping and eating habits, and generates advice for improving user sleep satisfaction.

### SUMMARY OF THE INVENTION

According to an aspect of some embodiments of the present invention there is provided a method of detecting sleep disturbances. The method comprises: sensing from a body part of a subject a subject signal indicative of a sleep state of the subject; sensing from an environment containing the subject an environment signal indicative of a state of the environment; identifying awakening and/or arousal events based on the subject signal, and changes in the environment state based on the environment signal. The method further comprises correlating the awakening and/or arousal events to the changes in the environment state, thereby detecting the sleep disturbances.

According to some embodiments of the invention the method comprises identifying at least one abnormal sequence of sleep stages based on the subject signal, and correlating between the abnormal sequence of sleep stages and the changes in the environment state.

According to some embodiments of the invention the method comprises dynamically controlling the environment state responsively to the correlation.

According to some embodiments of the invention the correlation comprises identifying a change in the environment state that precedes an awakening and/or arousal event within a time window of 1 minute or less, and defining the change in the environment state as a potential cause for the awakening and/or arousal event.

According to some embodiments of the invention the environment signal is indicative of ambient sound, wherein the identification of changes in the environment state comprises identifying a change in sound level which is above a predetermined threshold.

According to some embodiments of the invention the environment signal is indicative of ambient sound, wherein the identification of changes in the environment state comprises identifying a change in sound pitch.

According to some embodiments of the invention the environment signal is indicative of ambient light, wherein the identification of changes in the environment state comprises identifying a change in an amount of ambient light which is above a predetermined threshold.

According to some embodiments of the invention the environment signal is indicative of ambient temperature, wherein the identification of changes in the environment state comprises identifying a temperature change which is above a predetermined threshold.

According to some embodiments of the invention the environment signal is indicative of ambient temperature, wherein the identification of changes in the environment state comprises identifying when the ambient temperature crosses a predetermined ambient temperature threshold.

According to some embodiments of the invention the environment signal is sensed and the changes in the environment state are identified by a device contained in a single encapsulation.

According to some embodiments of the invention the environment signal is sensed by a sensor of a smartphone. According to some embodiments of the invention the identifying the awakening and/or arousal events is executed by a CPU of the smartphone. According to some embodiments of the invention the method further comprising transmitting the subject signal to the smartphone. According to some embodiments of the invention at least one of the identification of awakening and/or arousal events, the identification of the environment changes, and the correlation of the awakening and/or arousal events to the environment changes is executed by a CPU of the smartphone. According to some embodiments of the invention the subject signal is indicative of motion and is sensed by a sensor of a smartphone.

According to an aspect of some embodiments of the present invention there is provided a system for detecting sleep disturbances. The system comprises: a physiological sensor for sensing from a body part of a subject a subject signal indicative of a sleep state of the subject; an environmental sensor for sensing from an environment containing the subject an environment signal indicative of a state of the environment; and a data processor configured for identifying awakening and/or arousal events based on the subject signal, and changes in the environment state based on the environment signal. The data processor preferably correlates the awakening and/or arousal events to the changes in the environment state.

According to some embodiments of the invention the data processor is configured for identifying at least one abnormal sequence of sleep stages based on the subject signal, and for correlating between the abnormal sequence of sleep stages and the changes in the environment state.

According to some embodiments of the invention the system comprises a controller for dynamically controlling the environment state responsively to the correlation.

According to some embodiments of the invention the data processor is configured for identifying a change in the environment state that precedes an awakening and/or arousal event within a time window of 1 minute or less, and for defining the change in the environment state as a potential cause for the awakening and/or arousal event. According to some embodiments of the invention the time window is of 30 seconds or less.

According to some embodiments of the invention the environment signal is indicative of ambient sound, wherein the data processor is configured for identifying a change in sound level which is above a predetermined threshold.

According to some embodiments of the invention the environment signal is indicative of ambient sound, wherein the data processor is configured for identifying a change in sound pitch.

According to some embodiments of the invention the environment signal is indicative of ambient light, wherein the data processor is configured for identifying a change in an amount of ambient light which is above a predetermined threshold.

According to some embodiments of the invention the environment signal is indicative of ambient temperature, wherein the data processor is configured for identifying a temperature change which is above a predetermined threshold.

According to some embodiments of the invention the environment signal is indicative of ambient temperature, wherein the data processor is configured for identifying when the ambient temperature crosses a predetermined ambient temperature threshold.

According to some embodiments of the invention the environmental sensor and the data processor are contained in a single encapsulation.

According to some embodiments of the invention the environmental sensor is a sensor of a smartphone. According to some embodiments of the invention the data processor comprises a CPU of the smartphone.

According to some embodiments of the invention the system comprises a signal transmitter for transmitting the subject signal to the smartphone.

According to some embodiments of the invention at least one of the identification of awakening and/or arousal events, the identification of environment changes, and the correlation between the awakening and/or arousal events and the environment changes is executed by a CPU of the smartphone.

According to some embodiments of the invention the subject signal is indicative of motion.

According to some embodiments of the invention the subject signal is indicative of motion and the physiological sensor is a sensor of a smartphone.

According to some embodiments of the invention the subject signal is indicative of inter-beat-interval characterizing heart beats of the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of a system suitable for detecting sleep disturbances, according to some embodiments of the present invention;

FIG. 2 is a flowchart diagram of a method suitable for detecting sleep disturbances, according to various exemplary embodiments of the present invention;

FIG. 3 is a flowchart diagram describing a method suitable for identifying awakening and/or arousal events, according to some embodiments of the present invention;

FIG. 4 is a flowchart diagram describing a method suitable for determining REM sleep, according to some embodiments of the present invention;

FIG. 5 is a flowchart diagram describing a method suitable for determining one or more sleep stages other than REM, according to some embodiments of the present invention; and

FIG. 6 is a block diagram describing a data processing system according to some embodiments of the present invention.

### DESCRIPTION OF SPECIFIC EMBODIMENTS OF THE INVENTION

The present invention, in some embodiments thereof, relates to sleep analysis and, more particularly, but not exclusively, to a method and system for detecting sleep disturbances.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

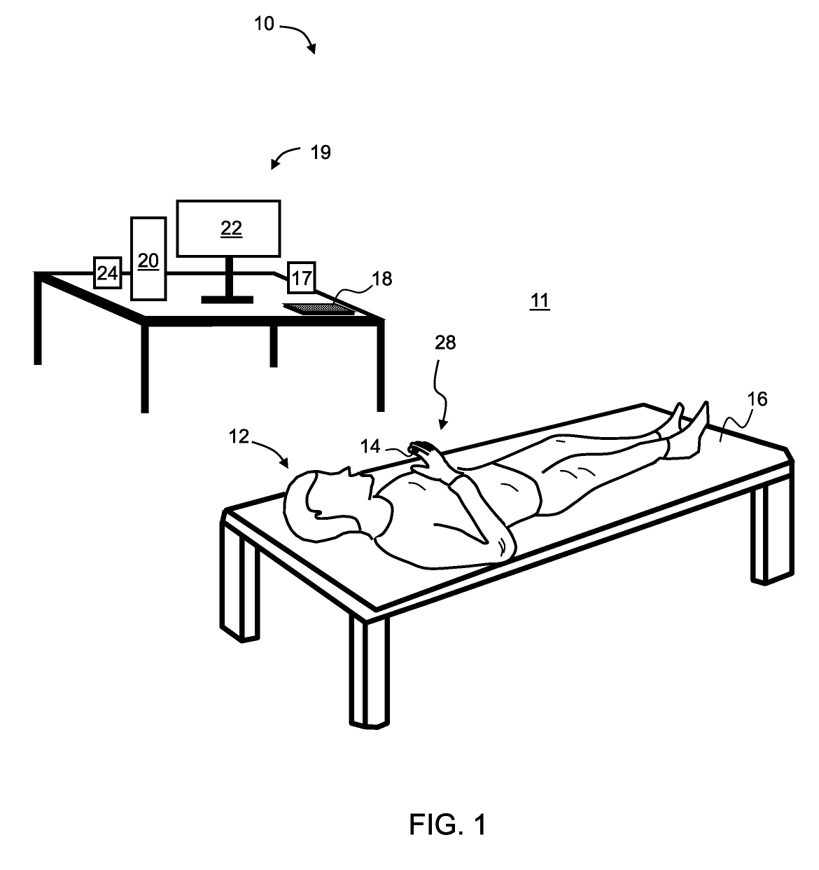

FIG. 1 is a schematic illustration of a system 10 suitable for detecting sleep disturbances, according to some embodiments of the present invention.

System 10 is particularly useful for monitoring a subject 12 during sleep, e.g., at home, hotel or any other sleeping location. System 10 may also be used in clinic or hospital ward environment or a dedicated sleep laboratory. System 10 receives and analyzes a subject signal from a suitable physiological sensor 14, and an environment signal from a suitable environmental sensor 18. Sensor 14 senses the subject signal from a body part 28 of subject 12, and sensor 18 senses the environment signal from an environment 11 containing subject 12. Environment 11 is typically a room in which subject 12 is sleeping. In various exemplary embodiments of the invention sensors 14 and 18 operate throughout the subject's sleeping time (e.g., throughout the night).

The subject signal provided by sensor 14 can be any type of signal that is indicative of a sleep state of subject. Preferably, the signal is indicative of the heart beats of subject 12, which are known to correlate well with sleep states. In various exemplary embodiments of the invention sensing sensor 14 converts mechanical, optical or electromagnetic waves into electrical signal. Representative of signals suitable for the present embodiments including, without limitation, an ECG signal, a blood pressure related signal, a blood volume related signal, an acoustic related signal (e.g., heart sound), a displacement related signal, and the like. Sensor 14 can thus be of any type provided it generates a signal, preferably electrical signal, such as one of the above signals.

Depending on the type of signal, physiological sensor 14 may or may not be physically attached to the subject.

For example, in some embodiments the subject signal is an ECG signal. In these embodiments sensor 14 the body part 28 is the chest of subject 12 and sensor 14 is attached to the chest, as known in the art of ECG monitoring.

In some embodiments of the present invention sensor 14 is a plethysmograph (e.g., photoplethysmograph) sensor which may be connected to one of the fingers on the hand of subject 12, as shown in FIG. 1, or elsewhere on the body of subject 12. In these embodiments, physiological sensor 14 provides a blood pressure or blood volume related signal.

In some embodiments of the present invention sensor 14 comprises a microphone which can be connected to a location on subject 12 such that the microphones generates an electrical signal in response to the sound of pulsatile blood flow in the subject's blood vessels or the sound of the heart itself, as known in the art. In these embodiments, physiological sensor 14 provides acoustic related signal.

In some embodiments of the present invention sensor 14 is a transducer/receiver device configured for detecting heart rate via radar technology. For example, the device can feature micro impulse radar pulses, wherein the distances between local maxima can be calculated and analyzed from the detected radar signal. Suitable transducer/receiver device is described in Florian Michahelles, Ramon Wicki, Bernt Schiele, “Less Contact: Heart-Rate Detection Without Even Touching the User,” iswc, pp. 4-7, Eighth IEEE International Symposium on Wearable Computers (ISWC'04), 2004, the contents of which are hereby incorporated by reference. The radar can optionally and preferably be a Doppler radar, particularly, but not exclusively, a non-contact Doppler radar. Doppler radars suitable for the present embodiments are described in U.S. Pat. Nos. 4,513,748 and 7,753,849 and references therein, the contents of which are hereby incorporated by reference.

In some embodiments of the present invention sensor 14 is a displacement, motion or vibration sensor (e.g., acoustic physiological sensor) which is connected to the body of subject 12 or to structure 16 supporting subject 12 (e.g., under the mattress), and which is configured for sensing vibrations of the subject's skin (directly or via structure 16) that are caused by the pulsatile blood flow in the vasculature of the subject. In these embodiments, physiological sensor 14 provides displacement related or acoustic related signal. Alternatively, sensor 14 can be an accelerometer which also provides data indicative of skin motion caused by blood flow. Motion, displacement and vibration sensors of sufficient sensitivity for sensing changes with such small amplitudes are known in the art and found, for example, in International Publication Nos. WO 00/67013, WO 03/036321, WO 03/048688, WO 2004/072658, WO 2005/062719, and WO2005/076727, and U.S. Pat. Nos. 6,984,993 and 7,080,554.

Additional techniques for sensing signals indicative of heart beats suitable for use in system 10 are known in the art and described, for example, in Reisner et al., Anesthesiology 2008, 108:950; Tazawa et al., The Auk, 1991, 108:594; and Verkruysse Otpics express 2008, 16: No 26, the contents of which are hereby incorporated by reference.

It is expected that during the life of a patent maturing from this application many relevant technologies for detecting signals indicative of heart beats will be developed and the scope of the term signal indicative of heart beats is intended to include all such new technologies a priori.

The environmental signal provided by sensor 18 can be any type of that is indicative of the state of environment 11. Preferably, environmental signal is indicative of at least one or at least two or at least three of ambient sound level, ambient sound pitch, ambient illumination, ambient temperature and ambient humidity. In some embodiments of the present invention the environmental signal is indicative of each of these ambient conditions. It is appreciated that when more than one type of ambient condition is sensed more than one environmental signal can generated. The term “environmental signal,” therefore, encompasses one or more signals pertaining to the state of the environment. Further, the term “environment sensor” encompasses a single sensor, a plurality of sensors contained in a single sensing system or an arrangement of sensors distributed in environment 11.

In some embodiments of the present invention environmental sensor 18 is a sensor of a smartphone.

As used herein “smartphone” refers to a cellular telephone with data processing functionality, and a programmable operating system.

Commercially available smartphones include many types of sensors, at least some of which can be utilized according to some embodiments of the present invention as environment sensor 18. Specifically, commercially available smartphones include a microphone, a camera image sensor, a temperature sensor, a humidity sensor, an accelerometer, a gyroscope, a GPS, a proximity sensor, an RFID, a touch screen gesture sensor, and other sensors. The system of the present embodiments contemplate using the microphone of the smartphone as a sound level and/or sound pitch sensor, the camera sensor as an ambient light sensor, the temperature sensor as an ambient temperature sensor and/or the humidity sensor as an ambient humidity sensor.

The use of a smartphone as a platform for environmental sensor 18 is advantageous from the standpoints of cost, simplicity and availability. The present embodiments also contemplated use of smartphone as a platform for sensor 14. For example, the accelerometer, microphone and/or touch screen gesture sensor can be used to provide a signal that is indicative of the heart beats of subject 12.

While some of the embodiments above are described with a particular emphasis to environmental sensor that employs a smartphone, other platforms for environmental sensor 18 are also contemplated. These other platforms include other types of personal gadgets (e.g., a tablet) or dedicated sensors deployed in environment 11. Representative examples of dedicated sensors including, without limitation, a stand-alone microphone for sensing sound level and/or sound pitch, a semiconductor or another type of light detector for sensing ambient illumination, a stand-alone temperature sensing device for sensing ambient temperature.

When the ambient illumination sensor is a camera (e.g., smartphone camera) that produces an image, for example, in the form of an array of picture-elements, the image is optionally and preferably reduced to a single parameter that represents the total intensity of the light over the image.

The sampling rate of the environmental signal is preferably at least 0.001 Hz, or at least 0.005 Hz, or at least 0.01 Hz or at least 0.05 Hz or at least 0.25 Hz or at least 1 Hz or at least 2 Hz or at least 4 Hz or at least 10 Hz. The sampling rate may also be different for different environmental parameters. Typically, the sampling rate is higher for ambient sound and ambient illumination than for ambient temperature. For example, the sampling rate for ambient sound and ambient illumination can be 1 Hz or higher, and the sampling rate for ambient temperature can be 0.05 or higher.

The subject signal from sensor 14 and the environmental signal from sensor 18 are optionally and preferably recorded for analysis. The analysis can be offline, or it can be executed during the collection of signals as described below. When the analysis is executed during the collection of signals, the signals can either be recorded or buffered for the analysis. The signals can be recorded by transmitting the signals to a data processor 20 using a receiver/transmitter 24.

The term “data processor” as used herein, includes any suitable device for processing data, including, without limitation, a microcomputer, a microprocessor, and a data processing system. A data processor can be electronic computing circuitry (e.g., a central processing unit) or a system associated with such circuitry. Representative examples include, without limitation, a desktop home computer, a workstation, a laptop computer and a notebook computer. Also contemplated is a dedicated system having electronic computing circuitry therein. Optionally, such a dedicated system is portable. Optionally, such a dedicated system is hand held or wearable, e.g., on the arm of the user. Also contemplated are systems which are capable of receiving and processing data but may also have other functions. Representative examples include, without limitation, a cellular telephone with data processing functionality, a personal digital assistant (PDA) with data processing functionality, a portable email device with data processing functionality (e.g., a BlackBerry® device), a portable media player with data processing functionality (e.g., an Apple iPod®), a portable gaming device with data processing functionality (e.g., a Gameboy®), and a tablet or touch screen display device with data processing functionality (e.g., an Apple iPad®).

Data processor 20 can receive the subject and environment signals using a wired communication line or via wireless communication (e.g., Bluetooth® communication, WiFi® communication, Infrared Data Association communication, home radio frequency communication etc.).

The data processor can receive the signals and store them on the processor's storage medium (e.g., hard drive) or transmit it for storage at a remote location such as a central server or a cloud facility. When sensor 18 is enacted by a smartphone, the environmental signal can be recorded by the CPU of the smartphone on the storage medium (e.g., SD card) of the smartphone, or it can be transmitted via the communication functionality of the smartphone to data processor 20 or to a remote location.

In some exemplary embodiments of the invention, the sensing of the environmental signal and subject signal are synchronized. This can be done by transmitting a synchronization signal simultaneously to sensors 14 and 18, one or more times during the subject's sleep. In some embodiments of the present invention a synchronization signal is transmitted to sensors 14 and 18 a plurality of times, e.g., before each sensing operation of the sensors. A synchronization signal can be transmitted by a dedicated controller 17 or by data processor 20 that can also serve as a controller.

In some embodiments, the synchronization between the signals is effected by a processor after the signals are recorded, in which case it is not necessary to transmit a synchronization signal to the sensors.

Data processor 20 may optionally be coupled, e.g., via a network receiver/transmitter 24 to communicate over a network, such as a telephone network or the Internet, with a remote data processor (not shown). This configuration allows detecting sleep disturbances while subject 12 is at a remote location (e.g., at home). Processor 20 can be a general-purpose processor (which may be embedded in a bedside, remote monitor or a dedicated system) with suitable software for carrying out the functions described below. This software may be downloaded to processor 20 in electronic form, or it may alternatively be provided on tangible media, such as optical, magnetic or non-volatile electronic memory. Alternatively, processor 20 can includes a dedicated circuitry constituted for carrying out the functions described below.

Any operation of processor 20 described below can alternatively be executed by a remote data processor, such as, but not limited to, a remote server or a cloud computing resource of a cloud computing facility. Still alternatively, any operation of processor 20 described below can be executed by the CPU of a smartphone.

Data processor 20 processes and analyzes the subject signal from physiological sensor 14 so as to identify one or more abnormal sequences of sleep stages. In various exemplary embodiments of the invention processor 20 identifies awakening and/or arousal events. Processor 20 optionally and preferably processes the signals in order to identify one or more sleep stages, including awakening and/or arousal events and periods. Optionally, the processor accesses a library of parameters during the processing. An example of a library which includes a dataset for characterizing sleep parameters is described in U.S. Pat. No. 7,623,912, the contents of which are hereby incorporated by reference. An exemplified procedure for identifying sleep stages is provided below.

Data processor 20 processes and analyzes the environmental signal from environment sensor(s) 18 so as to identify one or more changes in environment state.

When the environment signal is indicative of ambient sound, data processor 20 optionally and preferably identifies a change in sound level which is above a predetermined a sound level change threshold. Typical sound level change thresholds including, without limitation, 1 dB or 2 dB or 4 dB or 10 dB. Alternatively or additionally, data processor 20 can identify a change in sound pitch. For example, data processor can extract the frequency contents of the sound signal at two different, preferably adjacent, sampling times, compare the frequency contents, and identify a change in the pitch based on the comparison. Such a change may be manifested by the existence of one or more frequency band in the signal at one sampling time but not at the other sampling time. Such a change may also be manifested by a change of the sound intensity for a particular frequency band. A change of pitch can be declared when the intensity associated with a particular frequency band changes by a predetermined threshold (e.g., 1 dB or 2 dB or 4 dB or 10 dB).

When the environment signal is indicative of ambient light, data processor 20 optionally and preferably identifies a change in an amount of ambient light which is above a predetermined ambient light change threshold. Typical ambient light change thresholds include, without limitation, 0.1 lux or 0.5 lux or 1 lux or 2 lux or 4 lux or 10 lux.

There are several different types of ambient illumination changes that are contemplated. One type of ambient illumination change is an abrupt increase in the light intensity, such as the result of turning the lights on or opening of a door to a light flooded room. Another type of ambient illumination change is characterized by a slow increase in light intensity in environment 11 which then crosses an ambient light threshold (e.g., about 5 lux), this can be for example the result of the sunrise. Another ambient illumination change is characterized by fluctuation in the light intensity, such as the result of a TV set operating in environment 11.

When the environment signal is indicative of ambient temperature, data processor 20 identifies a temperature change which is above a predetermined temperature change threshold. Typical temperature change threshold include, in absolute value and without limitation, 1° C. or 2° C. or 3° C. or 4° C. or 5° C. Alternatively or additionally, data processor 20 can identify when the ambient temperature crosses a predetermined ambient temperature threshold T₀. Specifically, processor 20 can identifies when the temperature increases from below T₀ to above T₀ or decreases from above T₀ to below T₀. Typical values for T₀ include, without limitation, about 12° C. or about 14° C. or about 16° C. or about 18° C. or about 20° C. or about 22° C. or about 24° C. The present embodiments also contemplate use of a temperature band threshold, wherein processor 20 identifies when the ambient temperature is outside the temperature band threshold. A representative example of a temperature band threshold, includes, without limitation, a temperature band threshold of from about 12° C. to about 24° C.

The above thresholds for defining the environment state change may be predefined by system 10 or, more preferably, adjustable according to the typical depth of sleep of subject 12. Also contemplated are embodiments in which default thresholds are predefined, and the user is allowed to change those thresholds. In addition, environmental events can also be the outcome of more than one environmental input. In such case, the thresholds used for one environmental input may depend on inputs from another environmental signal.

Once the changes in environment state and the abnormal sequences of sleep stages and/or awakening and/or arousal events are identified, data processor 20 optionally and preferably correlates the changes in environment state to the abnormal sequences of sleep stages and/or awakening and/or arousal events.

The timing of each of the identified abnormal sequences of sleep stages and/or awakening and/or arousal events can define a time window Δt in which the data processor search for changes in the environment state that may have triggered the abnormal sequences of sleep stages and/or awakening and/or arousal events. An example of such a time window for sound signal may be the 30 seconds interval that precedes the beginning of an awakening and/or arousal event. Yet, for different types of changes in the state of the environment different durations of the time window may be employed, typically depending of the physiological reaction for each type of change. For example, sudden light or sound may cause an immediate awakening and/or arousal while gradual light change or temperature change may cause a relatively delayed awakening and/or arousal.

Thus, in various exemplary embodiments of the invention data processor 20 identifies an environment state change that precedes an awakening and/or arousal event within a predetermined time window Δt, and defines that environment state change as a potential cause for the awakening and/or arousal event. A typical value for the time window Δt is less than 1 minute or less than 30 seconds or less than 20 seconds or less than 10 seconds or less than 5 seconds or less. Longer time windows are also contemplated. For example, when the environment signals is indicative of the ambient temperature, the time window Δt is can be from about 5 minutes to about 60 minutes.

Data processor 20 can be a part of a console 19, which may include a display device 22. The results of the correlation can be used for issuing a report which is optionally and preferably displayed on a display device 22, such as, but not limited to, a computer monitor or the like, to present the results of the analysis to the user (for example, subject 12 himself or herself). Alternatively or additionally, data processor 20 may also be provided with an embedded display in which case the results can be displayed on the embedded display. Subject 12 can then use the displayed information for reducing the number of awakening and/or arousal causes. The results can also be transmitted to a computer readable medium for storage. Also contemplated are embodiments in which the results of the analysis are transmitted to a remote location where the results can be displayed and/or stored as desired.

In some embodiments of the present invention controller 17 dynamically controls the state of environment 11 responsively to the correlation. Typically, controller controls the state environment 11 so as to at least partially undo the identified change. For example, when the identified change corresponds to a noisy appliance (e.g., an air conditioner or a fan) controller 17 can turn off the appliance, and when the identified change corresponds to sunlight entering environment 11 through a window, controller 17 can close a curtain for blocking the light. In embodiments in which the state of environment 11 is dynamically changed responsively to the correlation, the identification of the changes in the state of the environment state and the identification of the abnormal sequences of sleep stages and/or awakening and/or arousal events are preferably within 1 minute or within 30 seconds or within 10 seconds or within 1 second.

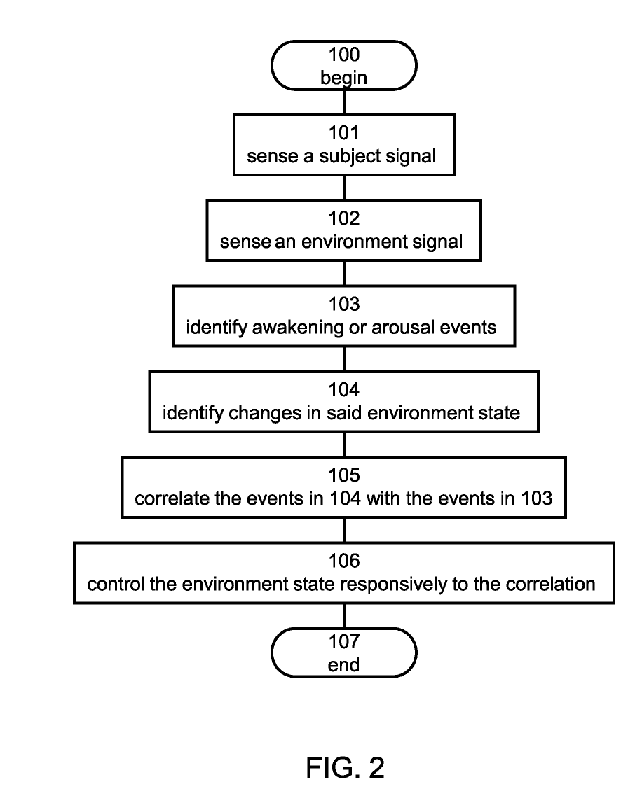

Reference is now made to FIGS. 2-5 which are flowchart diagrams of methods for analysis, according to various exemplary embodiments of the present invention.

It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

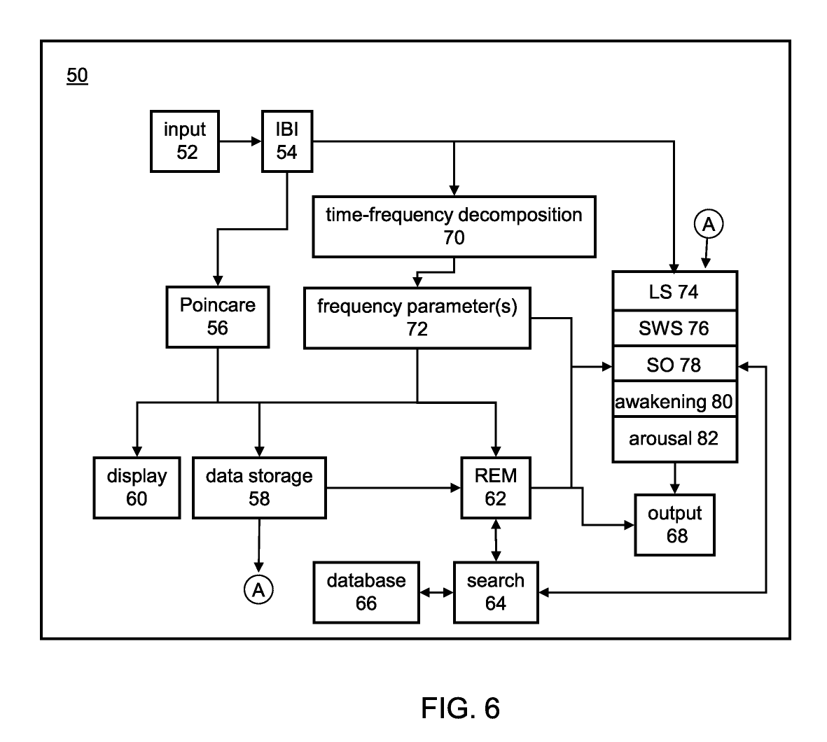

At least part of the operations described below can be implemented by a data processing system, e.g., a dedicated circuitry or a general purpose computer (e.g., processor 20), configured for receiving the signals and executing the operations described below. A representative and non-limiting example of a data processing system suitable for the present embodiments is described hereinunder with reference to the block diagram of FIG. 6.

Computer programs implementing the method of the present embodiments can commonly be distributed to users on a distribution medium such as, but not limited to, a floppy disk, a CD-ROM, a flash memory device and a portable hard drive. From the distribution medium, the computer programs can be copied to a hard disk or a similar intermediate storage medium. Alternatively, the computer programs can be downloaded to the hard disk or intermediate storage medium from a server, e.g., via the internet. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

The method of the present embodiments can be embodied in many forms. For example, it can be embodied in on a tangible medium such as a computer for performing the method operations. It can be embodied on a computer readable medium, comprising computer readable instructions for carrying out the method operations. It can also be embodied in electronic device having digital computer capabilities arranged to run the computer program on the tangible medium or execute the instruction on a computer readable medium.

FIG. 2 is a flowchart diagram of a method suitable for detecting sleep disturbances, according to various exemplary embodiments of the present invention. The method begins at 100 and continues to 101 at which a subject signal indicative of a sleep state of the subject is sensed, for example, using sensor 14 as further detailed hereinabove. The method continues to 102 at which an environment signal indicative of a state of the environment is sensed, for example, using sensor 18 as further detailed hereinabove. The method continues to 103 at which awakening and/or arousal events and/or other abnormal sequences of sleep stages are identified based on the subject signal, and to 104 at which changes in the environment state are identified based on the environment signal, as further detailed hereinabove. The method proceeds to 105 at which the changes in the environment state are correlated to the awakening and/or arousal events and/or other abnormal sequences of sleep stages, as further detailed hereinabove. The method optionally and preferably continues to 106 at which the environment state is dynamically controlled responsively to the correlation as further detailed hereinabove.

The method ends at 107.

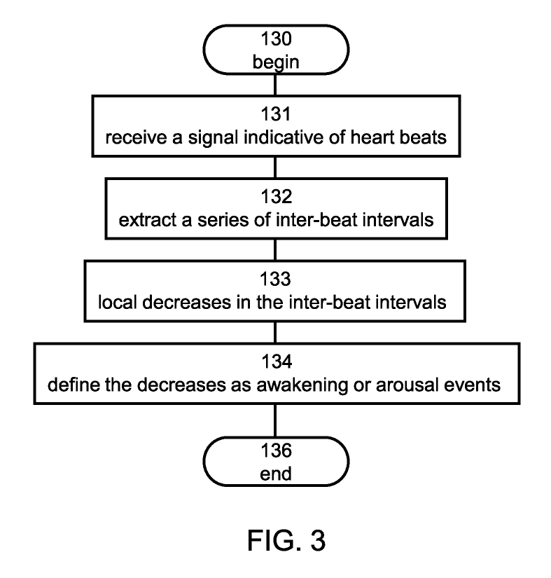

FIG. 3 is a flowchart diagram describing a method suitable for identifying awakening and/or arousal events, according to some embodiments of the present invention. The method begins at 130 and continues to 131 at which a signal indicative of heart beats of a subject, such as subject 12 (see FIG. 1) is received. The signal can be of any of the aforementioned types, and it can be input directly from a suitable physiological sensor (e.g., physiological sensor 14), or, following some pre-processing such as filtration, digitization and the like.

The method continues to 132 at which a series of inter-beat intervals (IBI) are extracted from the signal. In various exemplary embodiments of the invention the series of IBI is extracted from a single channel of the signal.

As used herein IBI refers to the duration of a heart beat on a beat by beat basis. The IBI is inversely proportional to the heart rate (HR).

The IBI can be extracted directly from the signal, for example, by identifying peaks in the signal and determining the duration between successive peaks. In some embodiments of the present invention the IBI is based on beat by beat values without any averaging. The advantage of this embodiment is that it allows identification of variability within the series. Thus, in this embodiment, the time resolution at which the IBI is extracted is preferably sufficiently high to reflect the beat-to-beat variability. In some embodiments of the present invention a 200 Hz resolution is employed. It is recognized that higher time resolutions correspond to signal of higher quality. Nevertheless, the present inventors discovered a correction procedure that can be employed for handling missing beats in the IBI signal. The correction procedure generally includes interpolation in order to close data time-gaps which are sufficiently short (e.g., time-gaps spanning over about 5 heart beats or less). When the data include longer time-gaps, the analysis can be performed in segmentation. The IBI series is optionally and preferably used for excluding spurious points from the data.

The method continues to 133 at which a local decrement in the IBI values is identified within the time domain.

As used herein “local decrement” refers to a time window during which the IBI values exhibit at least one decrement and do not exhibit any increment.

Local IBI decrement can be identified in more than one way.

In some embodiments of the present invention the method employs a running window, to search for a time interval at which all or most of IBI values are below a predetermined IBI threshold. This can be done by comparing the IBI values within the running window to IBI values corresponding to times that are earlier and/or later with respect to the running window. Use of times that are earlier with respect to the running window is particularly useful when the analysis is executed during the collection of signals. Use of times that are earlier as well as times that are later with respect to the running window is particularly useful for offline analysis. The IBI threshold is preferably expressed in terms of a percentile relative to the values that are earlier and/or later IBI values. It was found by the present inventors that a percentile in the range of 20-30 is suitable for identifying an IBI decrease. Once the time interval is identified it can be marked as a local IBI decrement or local IBI non-increment.

The width of the running window is typically from about 20 seconds to about 30 seconds. The width of the running window can also be defined in terms of heart beats. For example, the width of the running window can correspond to the duration of from about 10 to about 30 heart beats. The time period before and/or after the running window over which the percentile is calculated is typically from about 10 minutes to about 40 minutes, e.g., about 30 minutes.

In some embodiments of the present invention the method searches, e.g., using a running window, for a sequence of IBI values, preferably a sequence of adjacent IBI values that satisfies a predetermined criterion or set of criteria. One or more, preferably all, of the following criteria can be employed: (i) a sequence of at least N adjacent IBI values, where N is 5 or 10 or 15 or 20, (ii) the IBI values are monotonic within the sequence, (iii) the IBI values are non-increasing within the series, and (iv) the IBI values decreasing within the series.

The method continues to 134 at which each local non-increment or local decrement in the IBI decreases is defined as awakening or arousal event.

The method ends at 135.

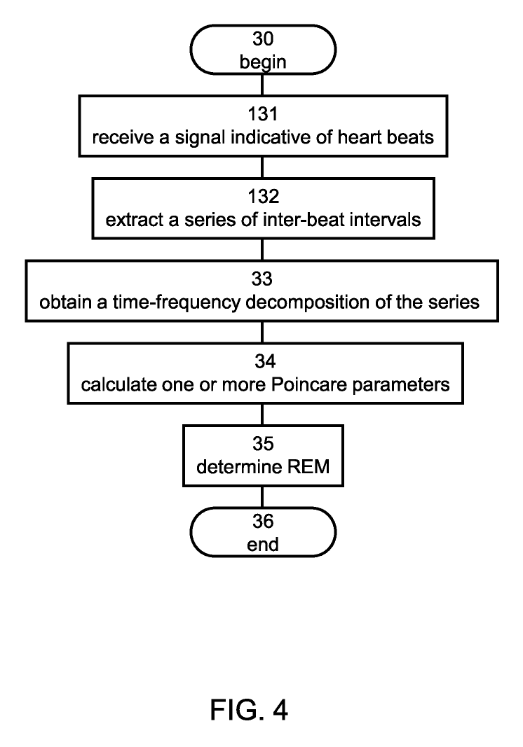

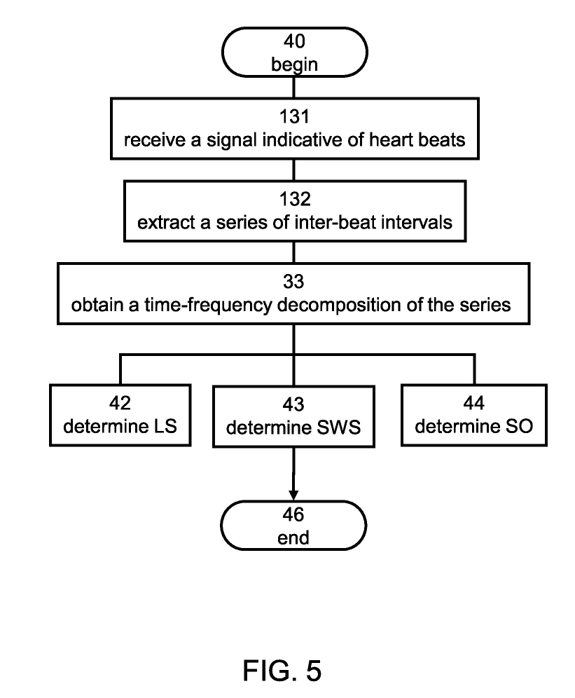

FIGS. 4 and 5 are flowchart diagrams of methods suitable for identifying various sleep states, where FIG. 4 describes a method suitable for determining REM sleep, and FIG. 5 describes a method suitable for determining one or more sleep stages other than REM.

Referring to FIG. 4, the method begins at 30 and continues to 131 at which a signal indicative of heart beats of a subject, such as subject 12 (see FIG. 1) is received, to as further detailed hereinabove. The method continues to 132 at which a series of inter-beat intervals (IBI) are extracted from the signal, as further detailed hereinabove.

In some embodiments of the present invention the method continues to 33 at which a time-frequency decomposition of the IBI series is obtained. The time-frequency decomposition may be obtained in any way known in the art for calculating the frequency content of a series. According to some embodiment of the present invention, the time-frequency decomposition is obtained by calculating at least one time-dependent power spectrum component. The power spectrum components include, but are not limited to, a very-low-frequency (VLF) power spectrum, a low-frequency (LF) power spectrum and a high-frequency (HF) power spectrum. A more detailed description of a procedure of obtaining the time-frequency decomposition is provided hereinunder.

As a general rule, the frequency bands reflect different activities of the autonomic nervous system. Specifically, high-frequencies reflect the fast reacting parasympathetic activity while low- and very-low-frequencies reflect both the parasympathetic and the slow reacting sympathetic activities.

The VLF power spectrum is typically defined for frequencies of from about 0.008 Hz to about 0.04 Hz, the LF power spectrum is typically defined for frequencies of from about 0.04 Hz to about 0.15 Hz, and the HF power spectrum is typically defined for frequencies of from about 0.15 Hz to about 0.5 Hz. According to some embodiments of the present invention, a combination of two or more of the above spectra is also calculated. For example, one power parameter may be the ratio LF/HF, and another power parameter may be the ratio VLF/HF. The ratio LF/HF is also known as the sympathovagal balance.

The method optionally and preferably continues to 34 at which one or more Poincare parameters are calculated from the IBI series. A Poincare parameter is a parameter that characterizes, at least partially, a Poincare plot. A Poincare plot is a graph generated from a vector of data. Typically, a Poincare plot is a two-dimensional graph in which a particular point on the graph represents a dependence of one datum ξ of the vector on a preceding datum η of the same vector, where the datum η (the preceding) may be referred to as “the cause” and the datum ξ may be referred to as “the effect”. In other words, the Poincare plot represents the dependence of a data set on its history. The gap between “the cause” and “the effect” may vary. According to some embodiments of the present invention, the gap is from about one heart-beat to about 10 heart-beats or more.

Although the Poincare parameters characterize a Poincare plot, it is not necessary to construct the plot in order for a particular Poincare parameter to be calculated. This is because some Poincare parameters can be calculated directly from the relation between elements of the IBI series, and some Poincare parameters are calculated while the plot is being constructed. Nevertheless, the construction of a Poincare plot before, during or after the calculation of one or more Poincare parameter is not excluded from the scope of the present invention.

Unless otherwise defined, the use of the term “plot” is not to be considered as limited to the transformation of data into visible signals. For example, a plot, such as a Poincare plot, can be stored as binary data (e.g., as a set of tuples) in a computer memory or any other computer-readable medium. Yet, in some embodiments of the present invention a plot can be transmitted to a display device such as a computer monitor, or a printer.

The method continues to 35 at which the Poincare plot is used for determining a REM sleep. This can be done in more than one way. In some embodiments of the present invention the method calculates the balance between odd and even quantiles (e.g., quartiles) in the Poincare plot, wherein the REM sleep is determined based on that balance. For example, when the balance is above a predetermined threshold the method determines that the subject is in REM sleep state, and otherwise the method determines that the subject is not in a REM sleep state. Alternatively, or additionally, the method can access an annotated library of data which comprises annotated values of such balance. In these embodiments, the method compares the calculated balance with the annotated balance values in the library until a matching balance is found, and determines whether or not the subject is in a REM sleep state based on the annotation associated with that balance.

The balance between odd and even quantiles is preferably calculated for a moving window in time in order to incorporate the variability of the balance during the time. A typical width of the window is, without limitation, about 60 seconds.

In some embodiments of the present invention the REM is determined based on a number of points in a vicinity of an identity line of Poincare plot. An identity line is a line defining all points for which the history datum is the same as the current datum. Typically, when the Poincare plot is visualized, such a line forms a 45° angle with the axes of the plot. The number of points in the vicinity of the identity line can be determine by defining on the plot a window of a predetermined width, associating the window with the identity line and counting the number of points falling in that window. A typical predetermined width of the window can be the time period spanned by a few (e.g., 2-5) sampling point. Thus, for example, when the absolute difference between two points is approximately the same or less than the time period spanned by a few sampling points the method can determine that both points are in the vicinity of the identity line.

The determination of REM sleep according to some embodiments of the present invention can be based on a thresholding procedure, wherein when the number of points in a vicinity of the identity line is above a predetermined threshold the method determines that the subject is in REM sleep state, and otherwise the method determines that the subject is not in a REM sleep state. Alternatively, or additionally, the method can access an annotated library of data which comprises annotated numbers of points in the vicinity of the identity line. In these embodiments, the method compares the counted number with the annotated number in the library until a matching number is found, and determines whether or not the subject is in a REM sleep state based on the annotation associated with that number.

The number of points in vicinity of the identity line is preferably calculated for a moving window in time in order to incorporate the variability of the parameter during the time. A typical width of the window is, without limitation, about 60 seconds.

The present embodiments also contemplate other Poincare parameters. In some embodiments of the present invention the Poincare parameters include one or more moments calculated for points of the Poincare plot which are selected within a predetermined time-widow (e.g., a two-minute time-window, a three-minute time-window, etc.).

Many moments are contemplated. One such moment is a moment of inertia. Broadly speaking, the moment of inertia is calculated by performing a summation of a plurality of squared distances of a plurality of points from a point, a line or a plane of reference, where each term in the summation is weighted by a respective mass. In one embodiment, all the points on the Poincare plot have equal “masses”. Hence, the moment of inertia is defined by IM=mΣDᵢ², where Dᵢ is a distance of the ith point of the Poincare plot from a predetermined line along the plot, m is an arbitrary mass parameter and the summation is over at least a portion of the points. The predetermined line is typically a straight line, such as, but not limited to, the aforementioned identity line.

Irrespective of the type of moments being chosen, the calculated moments may be normalized by dividing each moment by the total number of points. In addition, some of the points of the Poincare plot, failing to obey some statistical requirement, may be excluded from the calculation. For example, in embodiments in which the moments of inertia are used the statistical requirement may be that the distance, D, is smaller than the average of absolute D plus one standard deviation of D.

When the Poincare parameters include moments, the REM sleep can be identified when the respective moment which is below a predetermined threshold.

In some embodiments of the present invention the method uses one or more of the power spectra or frequency parameters as calculated from the time-frequency decomposition for determining the REM sleep. This can be done, for example, by accessing an annotated library of data which comprises annotated frequency parameters. In these embodiments the method compares the calculated frequency parameters with the annotated frequency parameters in the library until a matching parameter or set of parameters is found, and determines whether or not the subject is in a REM sleep state based on the annotation associated with that parameter or set of parameters.

Once the REM sleep is determined a report regarding the analysis can be issued, for example, by displaying the results on a display device and/or transmit them to computer readable medium. Optionally, the results can be transmitted over a network to a remote location at which they can be displayed and/or stored. The results of the analysis are optionally and preferably clustered over the time axis into a plurality of segments, each corresponding to one epoch of sleep. Thus, a cluster of instants at which REM sleep has been identified can be reported as an epoch at which the subject is in REM sleep, and a cluster of instants at which no REM sleep has been identified can be reported as an epoch at which the subject is not in REM sleep. A typical duration of an epoch is from about 10 seconds to about 60 seconds. Other durations are not excluded from the scope of the present invention.

The method ends at 36.

Referring to FIG. 5, the method begins at 40 and continues to 131 at which a signal indicative of heart beats of a subject is received as further detailed hereinabove. The method continues to 132 at which a series of IBI is extracted from the signal as further detailed hereinabove.

The method optionally and preferably continues to 33 at which a time-frequency decomposition of the IBI series is obtained, as further detailed hereinabove. The method then proceeds to at least one of 42, 43 at which the method uses one or more of the power spectra or frequency parameters as calculated from the time-frequency decomposition for determining a non-REM (NREM) sleep stage, such as, but not limited to, light-sleep (LS) and slow wave sleep (SWS). Optionally and preferably the method continues to 44 at which sleep-onset (SO) is determined.

The determination of LS, SWS and/or SO can be by a thresholding procedure or it can be based on a search in a library database. Also contemplated is a combination of the two approaches in which case an appropriate weight can be given to each approach.

SO is commonly referred to as a transition between quiet wakefulness and sleep. When a thresholding procedure is employed for determining SO, epochs corresponding to SO are preferably defined as being characterized by one or more SO parameters which is above a predetermined threshold, over a predetermined time range (typically 2-10 epochs). As the SO parameter(s) can be calculated by integrating one or more of the aforementioned power spectra over predetermined frequency limits. In some embodiments, the SO parameters are defined as time-dependent power ratios calculated using the integrated power spectra. The time-dependent power ratios may be, for example, a ratio between two integrated power spectra or a ratio between an integrated power spectrum and an integrated total power.

Beside integration limits which are the frequency thresholds defining the various power spectrum components, other integration limits may be used so as to optimize the ability of the SO parameters to characterize transition between quiet wakefulness and sleep.

One procedure for calculating the integration limits, according to some embodiments of the present invention, is by obtaining a steady state power spectrum from the IBI series and employing a minimum-cross-entropy method so as to separate between frequency peaks of the steady state power spectrum. The steady state power spectrum may be obtained by any known mathematical transform such as, but not limited to, a Fourier transform. The minimum-cross-entropy method is found, e.g., in the following publications, the contents of all of which are hereby incorporated by reference: Kullback, S., “Information Theory and Statistics”, John Wiley, New York, 1959; Seth, A. K., Kapur J. N., “A comparative assessment of entropic and non-entropic methods of estimation”, Maximum Entropy and Bayesian Methods, Fougere, P. F. (Ed.), Kluwer Academic Publishers, 451-462, 1990; Brink, A. D., Pendock N. E., “Minimum Cross-Entropy Threshold Selection”, Patt. Recog. 29: 179-188, 1996. The advantage of using the minimum-cross-entropy threshold method is that this method, without assuming any a priori knowledge about the original spectrum distribution, sets the optimal integration limits so that the difference in the information content between the original and segmented spectra is minimized.

In some embodiments of the present invention the SO parameter is normalized and/or analyzed by calculating a plurality of statistical quantities, such as, but not limited to, an average, a variance and a t-test.

When the determination of SWS is by thresholding, a predetermined threshold is optionally and preferably selected for separating SWS periods from NSWS periods. An epoch of SWS can be defined, for example, when a calculated power parameter is below a predetermined threshold. For example, a constant threshold may be imposed on the value of the LF power and/or the VLF power. A typical numerical value for this threshold is below the median (e.g., at about one third) of the possible range of the LF and/or VLF powers. Also contemplated, is a threshold which varies from one sleeping subject to another. For example, a power parameter may be averaged over the entire sleep of the sleeping subject. This average power parameter, which can be considered as a particular power average for the sleeping subject, may be used for choosing the threshold. For example, suppose that the power parameter is a ratio between LF power and HF power. Then, denoting the average of LF/HF for the entire sleep of the sleeping subject by <LF/HF>, the predetermined threshold is a function of <LF/HF>. The threshold may be a linear function of <LF/HF> where the parameters of linear function are determined from experimental measurements.

Optionally the threshold(s) also vary with time. In this embodiment, the numerical values of the above threshold is adapted to the overall tendency of power balance to change over the sleep. For example, if a constant threshold is used, this constant threshold is selected to be smaller during the beginning of the sleep and higher towards the end of the sleep. If the threshold is a function of some average power parameter (e.g., <LF/HF>), the parameters of the function are selected so that the value of the function is smaller during the beginning of the sleep and higher towards the end of the sleep.

A similar approach can be employed also for other sleep stages, e.g., LS. Alternatively, a combined procedure, which includes both the determination of REM sleep (e.g., by following the operations described above with respect to FIG. 4) and the determination of other sleep stages can be employed. For example, an epoch can be defined as corresponding to LS if it is an NSWS epoch other than a REM epoch and other than a SO epoch. Preferably, epochs corresponding to a non-sleep state are also excluded from being identified as being LS epochs.

Broadly speaking, non-sleep periods are accompanied first by an acceleration of the heart-rate (i.e., a decrement of the IBI values) and second by a deceleration of the heart-rate (i.e., an increment of the IBI values), where the IBI decrement is slower than its increment. In addition, before a non-sleep period the IBI values are typically above the IBI mean value. In various exemplary embodiments of the invention these characteristics are used for the purpose of determining the epochs of non-sleep periods from the IBI series.

There are different types of non-sleep periods occurring during sleep, which, according to a preferred embodiment of the present invention, can be determined by the method of the present embodiments. These include, but are not limited to, awakening periods and arousal periods. For a detailed definition of awakenings and arousals during sleep the reader is referred to an article by Bonnet M. et al., entitled “EEG arousals: scoring rules and examples: a preliminary report from the Sleep Disorders Atlas Task Force of the American Sleep Disorders Association”, published in Sleep, 15(2):173-84, 1992.

The main difference between awakenings and arousals is at the scale at which these non-sleep periods affect the signal. Specifically the awakening periods, which are typically characterized by trace duration of at least 15 seconds, affect the low frequencies region while the arousals periods, which are typically characterized by trace duration of 5-15 seconds, affect the intermediate-high frequencies region.

Thus, according to some embodiments of the present invention, the IBI series is filtered using a low-pass-filter thereby providing a first series of signals. Then, the awakening periods are defined as a plurality of epochs each associated with at least one of the first series of signals which is below a predetermined threshold.

Similarly, for the purpose of determining the arousal periods, the IBI series is optionally and preferably filtered using a band-pass-filter thereby providing a second series of signals. Then, the arousal periods are defined as a plurality of epochs each associated with at least one of the second series of signals which is below a predetermined threshold.

Typical thresholds for the awakening and arousals periods are about 0.85 of the averaged value of the first series and the second series of signals, respectively. A typical cutoff frequency for the low-pass-filter is about 0.01 Hz, and typical cutoff frequencies of the band-pass-filter are 0.05 Hz for the low limit and about 0.2 Hz for upper band limit.

The method ends at 46.

FIG. 6 is a block diagram describing a data processing system 50 suitable for executing various operations of the method described above. Data processing system 50 comprises a plurality of modules, each can be implemented as a dedicated circuitry within processor 20 of system 10 (see FIG. 1). Also contemplated, are embodiments in which one or more of the modules of data processing system 50 are tangibly embodied in a computer readable medium as computer program instructions, from which they can be loaded into the memory of a computer (e.g., a general purpose computer) for carrying out the respective operations. Further contemplated are embodiments in which some modules of system 50 are implemented as dedicated circuitry and some are embodied in a computer readable medium as computer program instructions.

System 50 comprises an input module 52 for receiving a signal indicative of heart beats of a sleeping subject. Input module 52 can be for example, for an A/D card when the signal is received directly from the physiological sensor, or an input data port such as a receptacle for a cable with a standard plug, such as a USB cable, or a dedicated plug, when the signal is a digital data stream. Input module 52 can also feature a wireless receiver in which case data is transmitted to input module using point to point wireless communication or broadcasted over a wireless communication network as known in the art. System 50 further comprises an IBI module 54 which receives the signal from input module 52 and extracts from a single channel of the signal a series of IBI, as further detailed hereinabove.

Optionally and preferably system 50 comprises a Poincare module 56 which calculates one or more Poincare parameters from the IBI series. Poincare module 56 optionally communicates with a data storage medium 58, which can be a computer memory or any other data writing device configured for writing data into a computer-readable medium. Any of the modules can communicates with a display device 60 such as a computer monitor or a printer, for displaying the quantity calculated by the respective module. In various exemplary embodiments of the invention system 50 comprises a REM module 62 which receives the Poincare parameter(s) from module 56 or medium 58 and determines the REM sleep of the subject based on the parameter(s), as further detailed hereinabove. REM module 62 is configured for extracting one or more of the aforementioned parameters from the Poincare plot. REM module 62 optionally and preferably communicates with a search module 64 which accesses a database 66 and searches it for matching parameters as further detailed hereinabove. The epochs identified as corresponding to REM sleep can be transmitted to an output module 68 which can be a display device and/or a receptacle for a cable with a standard or dedicated plug and/or a transmitter for wireless communication. Via output module 68, the identified epochs can also be stored in a computer readable medium.

In some embodiments of the present invention system 50 comprises a time-frequency decomposition module 70 which calculates a time-frequency decomposition of the IBI series as delineated hereinabove. A representative example of a procedure suitable for calculates a time-frequency decomposition is provided hereinunder. System 50 optionally and preferably comprises a frequency parameter module 72 which receive the time-frequency decomposition from module 70 and uses it for calculating one or more frequency parameters. Module 72 can also communicate with data storage module 58 for storing the calculated parameters, if desired. Optionally REM module 62 communicates with frequency parameter module 72 so as to allow determination of REM sleep based on the calculated frequency parameters. In various exemplary embodiments of the invention system 50 comprises at least one of a LS module 74, a SWS module 76, a sleep onset module 78, awakening module 80 and an arousal module 82. Modules 74, 76, 78, 80 and 82 receive the frequency parameters from module 72 and optionally also data from REM module 62 and determine LS, SWS, sleep onset, awakenings and/or arousals as further detailed hereinabove. Modules 74, 76, 78, 80 and 82 can also receive data (e.g., the frequency parameters) from data storage module 58. For clarity of presentation, data flow from storage module 58 to modules 74, 76, 78, 80 and 82 is shown via a block-diagram connector designated “A”. In some embodiments of the present invention one or more of modules 74, 76, 78, 80 and 82 communicates with search module 64 for the purpose of determining the respective state by comparison to entries in database 66. Epochs identified by one or more of modules 74, 76, 78, 80 and 82 as corresponding to the respective sleep can be transmitted to output module 68. Via output module 68, the identified epochs can be displayed, transmitted to a remote location and/or stored in a computer readable medium.

A detailed description of a method of obtaining the time-frequency decomposition, according to a preferred embodiment of the present invention, is now provided. The method, referred to herein as Selective Discrete Algorithm (SDA), was developed by Keselbrener L. and Akselrod S. and is found, e.g., in U.S. Pat. No. 5,797,840 and in an article entitled “Selective discrete Fourier transform algorithm for time-frequency analysis: Methods and application on simulated and cardiovascular signals” published in IEEE Trans. Biomed. Eng., 43:789, 1996, both of which are hereby incorporated by reference.

The SDA is a variable window method for time-dependent spectral analysis. This algorithm has been extensively validated on physiological signals (e.g., physiological signals in humans modulated by the ANS) under a variety of transient conditions. Generally speaking, the power spectrum of physiological signals in humans modulated by the ANS can be divided into the VLF range (below 0.04 Hz), the LF range (from 0.04 Hz to 0.15 Hz) and the HF range (above 0.15 Hz displaying a peak at about 0.2 Hz for adults and a peak at about 0.4 Hz for children). The HF range is mediated by the fast reacting parasympathetic nervous system, the LF range is mediated by both the parasympathetic nervous system and the slower reacting sympathetic nervous system and the VLF range is mediated by thermoregulation and unknown systems.

The SDA is directed at determining the power content of frequencies of interest embedded in the physiological signal. The essence of the SDA derives from a basic rule according to which the amount of information which is required to estimate the power of fluctuations is a decreasing function of the frequency of interest. More specifically, in order to estimate the power of a high frequency fluctuation, only a short string of data is required, while a low frequency fluctuation demands a much wider time window.

Hence, according to a preferred embodiment of the present invention, a selective windowed time-frequency (t-f) analysis is performed for providing the time-dependent power spectrum of the RRI series. For each time of interest and for each frequency of interest, a minimal time-window is chosen over the relevant digitized signal, as further detailed hereinbelow. According to a preferred embodiment of the present invention, a series of windows are generated along the signal within which the power spectrum of the frequencies under investigation is to be analyzed. Then, the power spectrum for a particular frequency within each window is determined.

According to a preferred embodiment of the present invention, the duration of the windows is generally a decreasing function of the frequency under investigation, preferably inversely proportional to the frequency. Hence, low frequencies are investigated using long time windows while high frequencies are investigated using short time windows. The t-f analysis can be at a wide range of resolutions, both in frequency and in time. Typically, the frequency resolution is in the order of 0.005 Hz at the low frequency end of the spectrum, with time resolution in the order of one minute. For the higher frequency end, frequency resolution is in the order of 0.02 Hz with time resolution of a few seconds. The time and frequency resolutions preferably reach intermediate values around the center of the t-f plane.

The selective windowed t-f analysis may be implemented by more than one way, for example, in one embodiment a wavelet transform is used, in another embodiment a selective discrete spectral transform is used, and the like.

In the embodiment in which wavelet transform is used, the aperture, duration and the time resolution between consecutive windows are defined by a prototype function h(t), a scale parameter, a, and a shift parameter, b, according to the wavelet transform ∫h_{ab}(t) f(t) dt. Further information on wavelet processing, is found in an article by Daubaechies I., entitled “The Wavelet Transform, Time Frequency Localization and Signal Analysis”, published in IEEE Transactions on Information Theory, Vol. 36. No. 5, 1990 the contents of which are hereby incorporated by reference.

As well known in the art, for a large scale parameter value, the prototype function is stretched such that the prototype wavelet acts as a low frequency function while, for a small scale parameter value, the prototype function is contracted such that the wavelet function acts a high frequency function. Hence, depending on the value assigned to scaling parameter, a, the wavelet function dilates or contracts in time, causing the corresponding contraction or dilation in the frequency domain. Thus, the wavelet transform provides a flexible time-frequency resolution and analyzes higher frequencies with better time resolution but poorer frequency resolution than lower frequencies.

In the embodiment in which a selective discrete spectral transform is used, a predetermined number of data points are selected from the windows. Based on the data points, the power spectrum of the frequency within the windows is calculated, using a mathematical transform, which may be, for example, a Fourier transform, a Haar transform, a Hartley transform, a sine transform, a cosine transform, a Hadamard transform, and the like. According to a preferred embodiment of the present invention the data points are selected by employing a low pass filter and undersampling technique such as moving average. Typically, the same number of data points is provided, irrespective of the duration of the windows, so as not to generate artifacts or normalization problems.

As mentioned hereinabove, the duration of windows is preferably inversely related to the frequency under investigation. Depending on the type of the selective windowed t-f analysis which is used, the duration of windows typically lies from about 2 periods to about 10 periods of the frequency under investigation. The windows can have different apertures including, but not limited to, a rectangular aperture, a Hamming aperture, a Hanning aperture, a Blackman aperture, a Gaussian window, a Lorentzian window, a sinc window, any power of a sine window, any power of a cosine window, any derivative of these windows, and the like.

Some corrections may be employed to the obtained power spectra, depending on the combination of the type of transform and the aperture of the window. For example, if the Fourier transform is used with a rectangular window, then, to ensure the highest possible frequency resolution by minimizing side lobes, the obtained power spectra are preferably corrected by dividing by the corresponding sinc function.

The calculated power spectra may be represented for example, in a 3D form, a 2D contour map form and the like. For example, if a power spectrum is represented by a 3D time dependent power spectrum graph, a first axis of the graph may represent frequencies, a second axis may represent time and a third axis may represent the power spectrum. Irrespective of the selected representation, the t-f resolution is substantially inhomogeneous, so that an optimal time-resolution is achieved for each frequency. Specifically, low frequencies have high frequency resolution and reduced time resolution, while high frequencies have lesser frequency and better time resolution.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification to are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

[1] Kim Y, Wilkens L R, Schembre S M, Henderson B E, Kolonel L N, Goodman M T. Insufficient and excessive amounts of sleep increase the risk of premature death from cardiovascular and other diseases: the Multiethnic Cohort Study. Prev Med. 2013, 57:377-85.
[2] Kita T, Yoshioka E, Satoh H, Saijo Y, Kawaharada M, Okada E, and Kishi R. Short sleep duration and poor sleep quality increase the risk of diabetes in Japanese workers with no family history of diabetes. Diabetes Care. 2012 February; 35(2): 313-318.
[3] Liu Y, Croft J B, Wheaton A G, Perry G S, Chapman P D, Strine T W, McKnight-Eily L R, and Presley-Cantrell L. Association between perceived insufficient sleep, frequent mental distress, obesity and chronic diseases among US adults, 2009 behavioral risk factor surveillance system. BMC Public Health. 2013; 13: 84.
[4] Miyata S, Noda A, Ozaki N, Hara Y, Minoshima M, Iwamoto K, Takahashi M, Iidaka T, Koike Y. Insufficient sleep impairs driving performance and cognitive function. Neurosci Lett. 2010, 469:229-33.
[5] Centers for Disease Control and Prevention (CDC). Drowsy driving—19 states and the District of Columbia, 2009-2010. MMWR Morb Mortal Wkly Rep. 2013 Jan. 4; 61:1033-7.
[6] Cappuccio F P, D'Elia L, Strazzullo P, Miller M A. Sleep duration and all-cause mortality: a systematic review and meta-analysis of prospective studies. Sleep. 2010, 33:585-92.
[7] Basner M, Muller U, Elmenhorst E M, Kluge K. and Griefahn B. Aircraft noise effects on sleep: a systematic comparison of EEG awakenings and automatically detected cardiac activations. Physiol. Meas. 2008, 29:1089-1103.
[8] Zeitzer J M, Dijk D J, Kronauer R E, Brown E N, Czeisler C A. Sensitivity of the human circadian pacemaker to nocturnal light: melatonin phase resetting and suppression. J. of Physiology 2000, 526:695-702.
[9] Gilbert S S, van den Heuvel C J, Ferguson S A, Dawson D, Thermoregulation as a sleep signalling system. Sleep Med Rev. 2004:8:81-93.
[10] Muzet A, Libert J P, Candas V. Ambient temperature and human sleep. Experientia 1984, 40:425-429.
[11] Haskell E H, Palca J W, Walker J M, Berger R J, Heller H C. The effects of high and low ambient temperatures on human sleep stages. Electroencephalogr Clin Neurophysiol. 1981, 51:494-501.
[12] Task Force on Sudden Infant Death Syndrome, Moon R Y. SIDS and Other Sleep-Related Infant Deaths: Expansion of Recommendations for a Safe Infant Sleeping Environment. Pediatrics. 2011 November; 128:1341-67.
[13] Reisner A, Shaltis P A, McCombie D, and Asada H H. Utility of the photoplethysmogram in circulatory monitoring. Anesthesiology 2008, 108:950.
[14] Verkruysse W, Svaasand L O, Nelson J S. Remote plethysmographic imaging using ambient light. Otpics express 2008, 16: No 26 (2003).

What is claimed is:

1. A method of detecting sleep disturbances, comprising:
sensing from a body part of a subject a subject signal indicative of a sleep state of said subject;
sensing from an environment containing said subject an environment signal indicative of a state of said environment;
identifying awakening and/or arousal events based on said subject signal, and changes in said environment state based on said environment signal; and
correlating said awakening and/or arousal events to said changes in said environment state, thereby detecting the sleep disturbances.

2. The method of claim 1, further comprising dynamically controlling said environment state responsively to said correlation.

3. The method of claim 1, wherein said environment signal is indicative of ambient sound and wherein said identifying said changes in said environment state comprises identifying a change in sound level which is above a predetermined threshold.

4. The method of claim 1, wherein said environment signal is indicative of ambient sound and wherein said identifying said changes in said environment state comprises identifying a change in sound pitch.

5. The method of claim 1, wherein said environment signal is indicative of ambient light, and wherein said identifying said changes in said environment state comprises identifying a change in an amount of ambient light which is above a predetermined threshold.

6. The method of claim 1, wherein said environment signal is indicative of ambient temperature, wherein said identifying said changes in said environment state comprises identifying a temperature change which is above a predetermined threshold.

7. The method of claim 1, wherein said environment signal is indicative of ambient temperature, wherein said identifying said changes in said environment state comprises identifying when said ambient temperature crosses a predetermined ambient temperature threshold.

8. The method of claim 1, wherein said environment signal is sensed and said changes in said environment state are identified by a device contained in a single encapsulation.

9. The method of claim 1, wherein said environment signal is sensed by a sensor of a smartphone.

10. The method of claim 1, wherein said subject signal is indicative of motion.

11. The method of claim 1, wherein said subject signal is indicative of inter-beat-interval characterizing heart beats of the subject.

12. A system for detecting sleep disturbances, comprising:
a physiological sensor for sensing from a body part of a subject a subject signal indicative of a sleep state of said subject;
an environmental sensor for sensing from an environment containing said subject an environment signal indicative of a state of said environment; and
a data processor configured for identifying awakening and/or arousal events based on said subject signal, and changes in said environment state based on said environment signal, and for correlating said awakening and/or arousal events to said changes in said environment state.

13. The system of claim 12, further comprising a controller for dynamically controlling said environment state responsively to said correlation.

14. The system of claim 12, wherein said environment signal is indicative of ambient sound and wherein said data processor is configured for identifying a change in sound level which is above a predetermined threshold.

15. The system of claim 12, wherein said environment signal is indicative of ambient sound and wherein said data processor is configured for identifying a change in sound pitch.

16. The system of claim 12, wherein said environment signal is indicative of ambient light, and wherein said data processor is configured for identifying a change in an amount of ambient light which is above a predetermined threshold.

17. The system of claim 12, wherein said environment signal is indicative of ambient temperature, and wherein said data processor is configured for identifying a temperature change which is above a predetermined threshold.

18. The system of claim 12, wherein said environment signal is indicative of ambient temperature, and wherein said data processor is configured for identifying when said ambient temperature crosses a predetermined ambient temperature threshold.

19. The system of claim 12, wherein said environmental sensor and said data processor are contained in a single encapsulation.

20. The system of claim 12, wherein said environmental sensor is a sensor of a smartphone.

21. The system of claim 12, wherein said subject signal is indicative of motion.

22. The system of claim 12, wherein said subject signal is indicative of inter-beat-interval characterizing heart beats of the subject.
